(19) United States
(12) Patent Application Publication
(10) Pub. No.: US 2007/0065470 A1
(43) Pub. Date: Mar. 22, 2007

(54) Oleaginous composition containing a flowable active ingredient

(76) Inventor: Dariush Behnam, Rossdorf (DE)

Correspondence Address: JACOBSON HOLMAN PLLC, 400 SEVENTH STREET N.W., SUITE 600, WASHINGTON, DC, 20004, US

(21) Appl. No.: 10/579,246

(22) PCT Filed: Nov. 15, 2004
(86) PCT No.: PCT/EP04/12945

§ 371 Date: May 12, 2006

(30) Foreign Application Priority Data
Nov. 14, 2003 (DE) 103 53 559.4

(51) Int. Cl. A61K 8/31; A23B 7/157

(52) U.S. Cl. 424/401; 426/262

(57) ABSTRACT

Described is an oleaginous composition containing an especially antioxidative, flowable active ingredient, said composition being used as an additive for an oil principally for food preparations, wherein the viscosity of the composition is between the viscosity of the active ingredient and the viscosity of the oil.

The invention relates to an oleaginous composition containing an especially antioxidative, flowable active ingredient, said composition being principally used for food preparations and also a method for the production of said composition.

The composition is added to an oil, which, for its part, serves for the preparation of food preparations or of salves and other cosmetics. Somewhat light vegetable oil, for example, rape oil, sunflower oil, safflower oil or even a middle-chained triglyceride such as miglyol, are taken into consideration as the oil. Active ingredients soluble in the oil against premature aging, especially antioxidants such as for example, ascorbic acid solubilised with an emulsifier (HLB value of between about 9 and 18) in accordance with EP 13 38 271, are added to these oils. Another active ingredient taken into consideration is micellated alpha lipoic acid in accordance with DE 101 08 614 A1. However, the capacity of the oil to absorb the added active ingredient is limited and not sufficient for many application cases. Thus, for example, a concentration of pure ascorbic acid in the oil of approximately 1,200 ppm is not exceeded for miglyol. Should the oil be used as an additive (compound) for dough pieces, it is desirable that a higher concentration of the active ingredient such as, for example, ascorbic acid be available in the oil.

Therefore the object underlying the invention is to increase the content of active ingredient in an oil suitable for food preparations or the like.

According to the invention, the viscosity of the composition mentioned at the onset is between the viscosity of the oil and the viscosity of the active ingredient. It is thus possible to substantially increase the active ingredient content in the oil. For this purpose, according to the invention, the active ingredient is first mixed with such a pre-mixture to form a composition in such a way that the composition has a viscosity, which is between the viscosity of the active ingredient and that of the oil; thereafter the composition is added to the oil. The advantage of the invention is that a substantially higher portion of the active ingredient can be incorporated into the oil than in case of a direct addition of the active ingredient to the oil. If the active ingredient is the mentioned ascorbic acid solubilisate, the portion of the pure ascorbic acid in the oil increases to approximately ten times with the present invention.

Suitable fats for the pre-mixture are oils such as, for example, fats rich in oil and/or in linoleic acid, fish oil or middle-chained trigylcerides. These are waxes, which consist of fat-like compounds, higher fatty acids and univalent higher paraffin alcohols (myricil alcohol, cetyl alcohol, deryl alcohol, melissyl alcohol), such as wool fat (E 913), beeswax or vegetable wax (carnauba wax, candelilla wax, sugar cane wax or even synthetic waxes). The wax variants such as wool wax, wool fat, artificial waxes or synthetically produced waxes mentioned as alternatives exhibit the desired viscosity-adjusting effect between the solubilisate and the oil only at higher concentrations. In case of carnauba wax, the homogeneity of the mixture can only be attained with difficulty. Variably large particles are formed in the process of cooling down. Therefore, cera alba proves to be a convenient agent for the purpose set forth in the present invention without limiting the invention to the use of this wax alone. The addition of any of the mentioned waxes to the fat of the pre-mixture generally amounts to only a small percentage by weight of the pre-mixture. For better dissolution of the wax in the fat, it can be recommended to prepare the pre-mixture under slight heat of for example, 40° C. to about 60° C. The active ingredient is added to the oil in the form of a composition, which consists of one part of the active ingredient and multiple parts of the pre-mixture. In the case of the mentioned micellated ascorbic acid, the composition expediently consists of one part of ascorbic acid solubilisate and approximately 9 parts of the pre-mixture.

The inventive method for the production of the composition provides for the active ingredient to be stirred with an oleaginous composition, which is adjusted in such a way that the viscosity of the composition is between the viscosity of the active ingredient and the viscosity of the oil.

Preferred embodiments of the inventive compositions and also expedient methods for the invention are specified in the dependent clauses.

The following is an example of a procedure for the production of the inventive oil: The viscosity of the individual mixtures at RT (20° C.) is measured as flow time in seconds in accordance with DIN 53211 using a FORD cup, which comprises a flow orifice of 3 mm. The flow time of the miglyol 812 used in both the following examples amounts to 31.5 seconds.

### EXAMPLE 1

In the first step, the pre-mixture B1 is prepared, which consists of cera alba (CA) and a light, especially vegetable fat, such as for example, sunflower oil, rape oil or safflower oil or alternatively neutral oil (miglyol 812). For this purpose, about 97.8% by weight (referred to 100% by weight of the pre-mixture) of the fat is mixed to 2.2% by weight of CA with one another and heated to about 40° C. to about 60° C. and stirred till the cera alba platelets are completely dissolved in the fat. The mixture is then cooled down to room temperature by stirring it constantly and slowly. The flow time of this pre-mixture amounts to 146.5 seconds.

In a second step, an ascorbic acid solubilisate A 1 is prepared separately, which consists of ascorbic acid, water and Polysorbate 20. For this purpose, 10% by weight of ascorbic acid is dissolved in 10% by weight (referred to 100% by weight of solubilisate) of distilled water under heat (40° C. to 60° C.) and added, while stirring it constantly at the specified temperature to 10% by weight of miglyol 812. Once the homogeneity of the mixture is attained, 70% by weight of heated Polysorbate 20 is incorporated and the mixture is stirred until it becomes clear and homogeneous. The flow time of this ascorbic acid solubilisate amounts to 867.0 seconds.

Thereafter, in a third step, 90% by weight of the pre-mixture B1 and 10% by weight of the ascorbic acid solubilisate A1 are mixed, stirring constantly, as a result of which the finished composition contains 1% by weight (=10,000 ppm) of pure ascorbic acid. The flow time of this composition amounts to 70.5 seconds.

### EXAMPLE 2

Preparation of a Pre-Mixture B2:

The pre-mixture B1 mentioned in example 1 contains an additive of a glyceride, for example, lamegin DWP 2000 (Cognis), which is a mono- and diacetyl tartaric acid ester of monoglycerides and diglycerides. In detail, 20% by weight of lamegin is added to 70% by weight (referred to the finished pre-mixture=100%) of the pre-mixture B1 and stirred until homogeneity is attained. The flow time amounts to 78.0 seconds.

The composition is then obtained by stirring 10% by weight (referred to the composition=100% by weight) of the ascorbic acid solubilisate A1 with 90% by weight of the pre-mixture B2 until it is homogenous. This composition contains 1% by weight (=10,000 ppm) of pure ascorbic acid. The flow time of the composition amounts to 85.0 seconds.

### EXAMPLE 3

Pre-mixture B3: 3.5% by weight of cera alba is added to 96.5% by weight (referred to the pre-mixture=100% by weight) of miglyol 812 at about 40° C. to about 60° C. and stirred under heat, till the cera alba platelets are completely dissolved. The hot mixture is cooled slowly to RT under constant stirring. Flow time: 115.5 seconds.

Solubilisate A3: About 20% by weight (referred to the solubilisate=100%) of ascorbic acid is dissolved completely in about 20% by weight of distilled water at about 45° C. About 60% by weight of Polysorbate 80 is added to the solution under stirring and heating to about 80° C. The solution is stirred until the solubilisate is clear and homogeneous.

Composition: About 5% by weight of the solubilisate A3 is stirred with about 95% by weight of the pre-mixture B3 until the composition is homogeneous. The composition then contains, in turn, about 1% by weight of pure ascorbic acid. Flow time: 179.5 seconds.

### EXAMPLE 4

Pre-mixture B4: About 96% by weight (referred to the pre-mixture=100%) of miglyol 812 is stirred under heat (example 3) with about 4% by weight of cera alba until the cera alba platelets are completely dissolved. The mixture is slowly cooled down to RT under stirring.

The composition is obtained by stirring about 10% by weight of the solubilisate A3 with about 90% by weight of the pre-mixture B4 until it is homogeneous. The composition contains on the whole about 2% by weight of pure ascorbic acid. The flow time amounts to 131.5 seconds.

It is apparent from the afore-mentioned examples that the viscosity of the respective composition, expressed in flow times, is between the viscosity of the solubilisate and that of the oil to be mixed with the composition. It is thereby important to take into consideration that the flow time of A3 cannot be determined according to the method used here due to the viscosity of A3 at RT. However, it can be assumed to be at least as high as that of A1.

The additional advantage of the invention described on the basis of the afore-mentioned examples, is that the sediment-free mixing of the oil with the inventive composition can be carried out at RT. However, the invention is not limited to the concrete numerical data and the mentioned starting materials. That is to say, if it can be tolerated that the addition of the composition to the oil can also take place under slight heat (about 30° C. to about 60° C.), even other pre-mixtures, solubilisates and compositions can be taken into consideration, as long as the viscosity of the composition at the selected temperature is between the viscosity of the solubilisate and that of the oil.

Therefore the following procedure is also possible: About 88.5% by weight (referred to the composition=100%) of an oil, which can be a vegetable oil, fish oil or a middle-chained triglyceride, is heated to about 80° C. About 1.5% by weight of beeswax (cera alba) is added to the hot oil and stirred. After the wax is dissolved, about 10% by weight of the afore-mentioned ascorbic acid solubilisate is added to the hot mixture. The stirring is continued until the composition has cooled down to room temperature after the heat is switched off.

This inventive composition remains steadily homogeneous and sediment-free even after longer periods of storage (at room temperature or thereunder) and contains approx. 1% by weight or 10,000 ppm of pure, chemically unchanged ascorbic acid (vitamin C). The concentration of this ascorbic acid in the oil is approx. 20 times more than the concentration, which is attained by derivatized ascorbic acid variants, such as e.g., ascorbyl palmitate (so far the only non-phenolic antioxidant variant) in oils.

The invention can also be implemented using an ascorbic acid solubilisate in which Polysorbate 20 is used instead of Polysorbate 80.

Instead of the afore-mentioned triglycerides, the ascorbic acid solubilisate can also be derived using middle-chained triglycerides, which usually comprise a substantial content of caprylic acid and caprinic acid. Furthermore, the ascorbic acid solubilisate can also be prepared by using Polysorbate 20 instead of Polysorbate 80, wherein the mentioned weight ratios can be substantially maintained. The flow time of such an ascorbic acid solubilisate prepared with Polysorbate 20 is measured to be 15.5 minutes.

The inventive composition can be added to the oil in a quantity according to requirements (up to 25% by weight). Thus the portion of ascorbic acid in the oil can be freely adjusted without additional measures (addition of additives or the use of special agitators) being necessary for this purpose.

As set forth herein, the composition is suitable for the conservation of oils. In addition, the composition can be used as the main component (oxidization compound) in the preparation of dough or dough mixtures, from which bakery products are to be prepared. Furthermore, the use of the composition as an anti-oxidative addition in the preparation of fish products, especially canned fish and as an additive for cosmetics is recommended.

1: An oleaginous composition containing an especially antioxidative, flowable active ingredient, as an additive for an oil principally used for food preparations, wherein the viscosity of the composition is between the viscosity of the active ingredient and the viscosity of the oil.

2: Composition according to claim 1, in which the active ingredient is present in a form in which it is micellated by an emulsifier having an HLB value of from 9 to 18.

3: Composition according to claim 1, in which the active ingredient is ascorbic acid, which is micellated using Polysorbate 20 or Polysorbate 80.

4: Composition according to claim 1, in which the oleaginous portion of the composition is the same oil as the one, to which the composition is added.

5: Composition according to claim 1, which consists of an oleaginous pre-mixture, which contains a small portion of wax, and the active ingredient stirred with the pre-mixture.

6: Composition according to claim 5, in which cera alba is used as wax.

7: Composition according to claim 5, in which the content of wax amounts to only a small percentage by weight of the pre-mixture.

8: Composition according to claim 1, consisting of about one part of active ingredient and multiple, especially nine parts of the pre-mixture.

9: Composition according to claim 1, in which the pre-mixture comprises about 1% by weight to about 4% by weight of wax, especially cera alba.

10: Method for the production of a composition according to claim 1, in which the active ingredient is stirred with an oleaginous pre-mixture, which is adjusted in such a way that the viscosity of the composition is between the viscosity of the active ingredient and the viscosity of the oil, to which the composition is added.

11: Method according to claim 10, in which the pre-mixture contains a small portion of wax.

12: Method according to claim 11, in which the wax is stirred with the oil portion of the pre-mixture under heat, for example about 40° C. to about 60° C.

13: Method according to claim 11, in which lamegin or similar emulsifiers are added to the pre-mixture.

14: Method according to claim 11, in which cera alba is used as the wax.

15: Method according to claim 11, in which the oleaginous portion of the pre-mixture is stirred with about 1% by weight to about 4% by weight of wax, especially cera alba.

16: Method according to claim 10, in which about one part of active ingredient is stirred with multiple, especially nine parts of the pre-mixture.

17: Use of the composition according to claim 1 as an additive for an oil to be added to dough pieces or bakery products or fish products or to a cosmetic.
